(19) United States
(12) Patent Application Publication
(10) Pub. No.: US 2021/0030918 A1
(43) Pub. Date: Feb. 4, 2021

(54) COATING OF FIBERS WITH DIPEPTIDE NANOSTRUCTURES USING ULTRASONIC CAVITATION

(71) Applicant: Ramot at Tel-Aviv University Ltd., Tel-Aviv (IL)

(72) Inventors: Lihi ADLER-ABRAMOVICH, Tel-Aviv (IL); Ehud GAZIT, Tel-Aviv (IL); Irena GRIGORIANTS, Tel-Aviv (IL); Lialy KHADEJA, Tel-Aviv (IL); Sharon GILEAD, Tel-Aviv (IL)

(73) Assignee: Ramot at Tel-Aviv University Ltd., Tel-Aviv (IL)

(21) Appl. No.: 16/942,837

(22) Filed: Jul. 30, 2020

Related U.S. Application Data
(60) Provisional application No. 62/880,686, filed on Jul. 31, 2019.

(51) Int. Cl. A61L 15/32; A61K 31/12; A61L 15/42; D06M 10/02; D06M 15/01; D04H 13/00

(52) U.S. Cl. CPC A61L 15/32; A61K 31/12; A61L 15/425; D06M 2101/06; D06M 15/01; D04H 13/00; D06M 10/02

(57) ABSTRACT

Provided herein are composite structures that include a core fiber and a plurality of peptide-based self-assembled nanostructures attached thereto, wherein the nanostructures may be loaded with a bioactive agent, such that the composite structures may act as slow-release drug-delivery medical device. Also provided is process for producing the composite structures, and uses thereof.

### RELATED APPLICATION(S)

This application claims the benefit of priority under 35 USC § 119(e) of U.S. Provisional Patent Application No. 62/880,686 filed on Jul. 31, 2019, the contents of which are all incorporated by reference as if fully set forth herein in their entirety.

### FIELD AND BACKGROUND OF THE INVENTION

The present invention, in some embodiments thereof, relates to drug-releasing medical device, and more particularly, but not exclusively, to a fabrics and fibers coated with a drug-releasing dipeptide nanostructures.

Cotton fabric containing motley cellulose, has been widely used for centuries. In recent years, non-woven fabrics, which are engineered, polymer-based fabrics, replace some of the cotton applications, for example as disposable fabrics. In biomedicine applications, both cotton and non-woven fabrics are being used as wound dressings, bandages, surgical tapes and meshes, uniforms and tissue scaffolds. For these performances, the fabrics are often modified to create functions such as antimicrobial activity, conductivity and ultraviolet protection or illumination. Functionalized fabrics are often produced by the integration of nanomaterials, however their integration remains challenging due to their lack of ability to form covalent bonds to fix them onto the fabric surface. Great effort has been made to incorporate therapeutic materials into the fabrics that can be released in a slow manner, in particular, nano metal oxides, such as silver and zinc, for antibacterial applications.

Various techniques such as impregnation process, pad-dry-cure method, and layer by layer assembly have been adopted to prepare fabrics coated or incorporated with nanoparticles. Nevertheless, the manufacture of durable fabrics incorporated with nanoparticles on an industrial scale is still a great challenge. An alternative method for direct deposition and insertion of nanoparticles into fabrics is sonochemical method [Perelshtein, I. et al., Nanotechnology, 2008, 19, 245705]. It has been previously shown that coated nylon, polyester and cotton fabrics with silver nanoparticles using this method demonstrated antibacterial properties. In addition, colloidal silver were shown to coat paper using ultrasonic radiation. The advantages of this method are the short-time procedure, involving one-step/pot reaction and the lack of toxic chemical presence. In this process, ultrasonic waves produce acoustic cavitation, i.e. the formation, growth and implosive collapse of bubbles in a liquid [Suslick, K. S., Science, 1990, 247, 1439-1445]. Upon cavitational collapse, an extremely high temperature (5000-25000 K) and pressure (1000 atm) are achieved on a minute area for nanoseconds, these extreme conditions permit exclusive chemical and physical effects at this pointed area. The containing products are microjetted towards the fabrics' fibers at high speed, leading to their physical adherence and even penetration to the surface of the fibers.

Silva R. et al. [Biotechnol. J., 2012, 7, 1376-1385] proposed a methodology for the controlled release of active components for the healing of burn wounds. Cotton and non-woven bandages have been cationised to promote the attachment of protein microspheres, and the active agents, piroxicam and vegetable oil, were entrapped into the microspheres using ultrasound energy. Active agents were released from the microspheres by a change in pH. Wound healing was assessed using standardized burn wounds induced by a cautery in human full-thickness skin equivalents (EpidermFT). The best re-epithelialization and fastest wound closure was observed in wounds treated with proteinaceous microspheres attached to gauzes, after six days of healing, in comparison with commercial collagen dressing and other controls. Furthermore, the ability of these materials to reduce the inflammation process, together with healing improvement, suggests these biomaterials are suitable for wound-dressing applications.

Dipeptide nanotubes have been widely investigated as next-generation biomaterials for various chemical, technological, engineering, and drug delivery applications [Gazit, E., Chem. Soc. Rev. 2007, 36, 1263-1269]. Diphenylalanine (FF), an aromatic dipeptide, is of particular interest for industrial applications as its short chain length makes its synthesis and manufacturing upscale more effective. The FF dipeptide nanotubes were shown to have antibacterial activity which is sufficient to eradicate mature biofilm forms of bacteria widely implicated in hospital infections [L. Porter, S. et al., Acta Biomater. 2018, 77, 96-105]. The FF nanotubes completely inhibit bacterial growth, trigger upregulation of stress-response regulons, induce substantial disruption to bacterial morphology, and cause membrane permeation and depolarization. It has been shown that cationic dipeptide, H-Phe-Phe-NH₂, can self-assembled to form nanotubes that upon dilution can rearranged to form vesicles. The cationic dipeptide were further showed to encapsulate single strand DNA and could enter cells cytoplasm. The aromatic dipeptide N-fluorenylmethoxycarbonyldi-phenylalanine were shown to form hydrogel nanoparticles for controlled drug delivery. These nano-carriers were formed using inverse emulsion technique and were louded with the antitumor drugs doxorubicin and 5-flourouracil and showed release kinetics of the drugs. Additional antitumor drug, hydroxycamphothecin, has been delivered to cells through the internalization of the self-assembled nanofibers to which it was bound, with a sustained drug release over a one-week period. These dipeptides were composed of D-amino acids that provided excellent protease stability, allowing for prolonged therapeutic effect as observed in vivo over several weeks, with reduction of a tumor mass in a rat model.

International Patent Application Publication No. WO2014/132262, by the present assignee, teach compositions comprising self-assembled nanotubes formed of short peptides which comprise one or more aromatic amino acid residue(s) in an inverted emulsion are disclosed. Such nanotubes which encapsulate an active agent and uses thereof in therapeutic and diagnostic applications are also disclosed therein.

International Patent Application Publication No. WO2014/178057, by the present assignee, teach ordered (e.g., self-assembled) structures, arranged from peptide nucleic acids and/or analogs thereof. The peptide nucleic acids forming the ordered structures comprise from 1 to 10 PNA backbone units, at least one comprising a guanine nucleobase or an analog thereof. Processes of generating the ordered structures, uses thereof and articles-of manufacturing, devices and systems containing same are also disclosed therein.

International Patent Application Publication No. WO2017/068584, by the present assignee, teach ordered structures composed of a plurality of self-assembled peptide nucleic acid (PNA) monomers, and processes of generating same. The plurality of PNA monomers includes modified PNA monomers which are N-protected PNA monomers and/or which feature at least one aromatic moiety attached to a backbone, a nucleobase and/or a nucleobase linkage unit of the PNA monomer. Tunable photonic crystals formed of the provided ordered structures, uses thereof and articles-of-manufacturing containing same are also provided therein.

International Patent Application Publication No. WO2019/012545, by the present assignee, teach hybrid hydrogels, made of a three-dimensional network of fibrillar nanostructures, at least a portion of the fibrillar nanostructures being formed of at least two different types of aromatic moieties, at least one type of the aromatic moieties being an end-capping modified aromatic dipeptide and at least another type of the aromatic moieties being an amine-modified halogenated aromatic amino acid, are provided. Also provided therein are processes of preparing the hybrid hydrogels and uses thereof.

U.S. Patent Application Publication No. 2011/0300767 discloses a system for preparing fabrics with antibacterial properties by sonochemically impregnating the fabrics with proteinaceous microspheres loaded with antibiotic.

Additional prior art documents include EP2294260, EP2839070, Abramov, O. V. et al., Surface & Coatings Technology, 2009, 204, 718-722, Petkova. P. S. et al., ACS Appl. Mater. Interfaces, 2014, 6, 1164-1172, and Tzhayik, O. et al., Ultrasonics Sonochemistry, 2017, 38, 614-621.

### SUMMARY OF THE INVENTION

Aspects so the present invention are drawn to providing a solution to the problems associated with large-scale production of functionalized textiles, e.g., for use as medical devices. Provided herein, according to some embodiments of the present invention, are diphenylalanine (FF) dipeptide nanotubes, deposited into cotton and non-woven fabrics using sonochemical irradiation. According to some embodiments, the FF nanotubes are further loaded with a bioactive agent, such as curcumin, and are shows to exhibit a sustained release thereof from the fabrics. The structure of the nanotubes-fabric interactions is shown by electron microscopy and the sustained release of the bioactive agent is demonstrated using absorbance measurements. Following sonication, the nanotubes are fully embedded onto the fabric fibers and enabled sustained release of a bioactive agent. The results presented in the Examples section demonstrate the broad utility of the presently provided method for large scale fabrication of functionalized commercial fabrics.

Thus, according to an aspect of some embodiments of the present invention there is provided a composite structure that includes a core fiber and a plurality of peptide-based self-assembled nanostructures (SAPBNSs) attached thereto.

According to some embodiments of the invention, the SAPBNSs comprise at least one peptide having 2-6 amino-acid residues.

According to some embodiments of the invention, the peptide includes at least one aromatic amino acid residue.

According to some embodiments of the invention, the peptide is a dipeptide.

According to some embodiments of the invention, the dipeptide is a homodipeptide.

According to some embodiments of the invention, the homodipeptide is selected form the group consisting of phenylalanine-phenylalanine dipeptide (diphenylalanine peptide; FF), naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide.

According to some embodiments of the invention, the nanostructures are essentially nanotubes.

According to some embodiments of the invention, the core fiber is a cotton fiber, a wool fiber, a silk fiber, a synthetic fiber and a mixed natural and synthetic fiber.

According to some embodiments of the invention, the SAPBNTs are attached to the core fiber as assessed by being substantially resistant to reparative washing by an aqueous solution.

According to some embodiments of the invention, the SAPBNTs include a bioactive agent engaged therewith.

According to some embodiments of the invention, the bioactive agent is curcumin.

According to another aspect of some embodiments of the present invention there is provided a fabric that includes at least one fiber provided herein.

According to some embodiments of the invention, the fabric is a woven fabric.

According to some embodiments of the invention, the fabric is a non-woven fabric. According to some embodiments of the invention, the fabric is fabric provided herein consists of the fiber provided herein.

According to another aspect of some embodiments of the present invention there is provided a medical device that includes the fabric provided herein, or at least one fiber provided herein.

According to some embodiments of the invention, the medical device is, optionally, a gauze, a wound dressing, a stitching thread, a bandage or a mesh.

According to yet another aspect of some embodiments of the present invention there is provided a process of producing the fiber provided herein, or the fabric provided herein, the process is effected by:

providing the plurality of peptide-based self-assembled nanostructures in a solution;

contacting the solution that includes the plurality of self-assembled peptide-based nanostructures with the fiber or the fabric; and

subjecting the solution to sonochemical (ultrasound) irradiation.

According to some embodiments of the invention, the step of providing SAPBNSs is effected by:

mixing the peptide in an aqueous medium;

heating the medium to 70-95° C. for at least 20 minutes while mixing; and

cooling the medium.

According to some embodiments of the invention, the step of mixing the peptide in an aqueous medium, further includes adding a bioactive agent to the aqueous medium.

According to some embodiments of the invention, the sonochemical irradiation is effected at the energy level that ranges from 40 watt to 400 watt.

According to some embodiments of the invention, the concentration of the SAPBNSs in the solution ranges 1-5 mg/ml

According to some embodiments of the invention, the step of subjecting the solution to sonochemical (ultrasound) irradiation is effected over a time-period that ranges from 1 second to 10 minutes.

According to another aspect of some embodiments of the present invention there is provided a method of treating a medical condition using the medical device provided herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a schematic illustration of the process 10 of functionalization of fabrics, according to some embodiments of the present invention, wherein dipeptide FF 11 is incubated at 90° C. and cooled to 25° C. so as to form nanotubes 12, which are then subjected to ultrasound waves 13, emanating from ultrasound probe 14 in the presence of fabric 15;

FIGS. 2A-D present an optic microscope image of FF dipeptide nanotubes (FIG. 2A), a SEM micrograph of FF dipeptide nanotubes (FIG. 2B), a SEM micrograph of FF dipeptide nanotubes deposited on non-woven fabric using sonication irradiation (FIG. 2C), and a SEM micrograph of FF dipeptide nanotubes deposited on cotton fabric using sonication (FIG. 2D);

FIGS. 3A-F present SEM images showing the durability of the nanostructure, coated by sonication, to the fabrics, wherein uncoated non-woven fabrics are shown in FIG. 3A, non-woven fabrics coated by FF nanotubes using sonication irradiation are shown in FIG. 3B, non-woven fibers coated by FF nanotubes using sonication irradiation, after washings are shown in FIG. 3C, uncoated cotton fabrics are shown in FIG. 3D, cotton fabrics coated by FF nanotubes using sonication irradiation are shown in FIG. 3E, and cotton fibers coated by FF nanotubes using sonication irradiation, after washings are shown in FIG. 3F;

FIGS. 4A-F present SEM images showing the correlation between the degree of SAPBNTs coating of core fibers in synthetic non-woven fabrics as a function of sonochemical (ultrasound) irradiation time, wherein FIG. 4A shows an unmodified synthetic non-woven fabric, FIG. 4B shows the synthetic non-woven fabric with absorbed nanotubes without sonication, FIGS. 4C-F show the synthetic non-woven fabric coated with nanotubes following sonochemical irradiation duration of 30 seconds (FIG. 4C), 1 minute (FIG. 4D), 3 minutes (FIG. 4E), and 5 minutes (FIG. 4F);

FIGS. 5A-F present SEM images showing the correlation between the degree of SAPBNTs coating of core fibers in 100% cotton fabrics as a function of sonochemical (ultrasound) irradiation time, wherein FIG. 5A shows an unmodified cotton fabric, FIG. 5B shows the cotton fabric with absorbed nanotubes without sonication, and FIGS. 5C-F show the cotton fabric coated with nanotubes following sonochemical irradiation duration of 30 seconds (FIG. 5C), 1 minute (FIG. 5D), 3 minutes (FIG. 5E), and 5 minutes (FIG. 5F);

FIGS. 6A-D present SEM micrographs showing optic microscope image of curcumin encapsulation in FF nanotubes (FIG. 6A), fluorescence microscopy of curcumin encapsulated in FF nanotubes (FIG. 6B), non-woven fabric deposited with curcumin loaded FF nanotubes observed by SEM (FIG. 6C), and cotton fabric deposited with curcumin loaded FF nanotubes observed by SEM (FIG. 6D); and

FIGS. 7A-B present curcumin release kinetics profiles, as observed while released from dipeptide nanotubes attached to the fabrics, according to some embodiments of the present invention, wherein absorbance kinetics of curcumin released from FF nanotubes coated non-woven fabric (430 nm) is shown in FIG. 7A, and slow release graph of curcumin from cotton coated by FF nanotubes is shown in FIG. 7B, whereas the curves were normalized according to the calibration curves of curcumin solution.

### DESCRIPTION OF SOME SPECIFIC EMBODIMENTS OF THE INVENTION

The present invention, in some embodiments thereof, relates to drug-releasing medical device, and more particularly, but not exclusively, to a fabrics and fibers coated with a drug-releasing dipeptide nanostructures.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While contemplating the present invention, the inventors have considered harnessing the benefits and utility of self-assembled peptide-based nanotubes as effective drug-delivery vehicle incorporated into fibers or fabrics. The incorporation of such nanostructures by sonochemistry was ill-advised, since sonochemistry involves extremely high temperatures (5000-25000 K) and pressure (1000 atm) occurring over a very small area for nanoseconds, and the expectations was that these extreme conditions would destroy the nanostructures; hence, the as the expectation was that the nanostructures would not sustain the ultrasound irradiation, and instead of being implanted into the fiber, the nanostructures would collapse. The present inventors have surprisingly found that self-assembles peptide-based nanostructures are able to substantially maintain their structural integrity in the sonication process, as well maintaining their capacity to encapsulate bioactive agents or drugs into their structures and controllably release them.

While reducing the present invention to practice, the inventors have successfully deposited diphenylalanine dipeptide (FF) nanotubes on cotton and non-woven fabrics using sonochemical irradiation. FF dipeptide nanotubes were further loaded with curcumin, an antioxidant, antimicrobial, and anti-inflammatory agent. The structure of the nanotubes-fabric interactions was studied by electron microscopy, and the sustained release of curcumin was demonstrated using absorbance measurements. Sonication duration was positively correlated to the deposition of the nanotubes onto the fabrics' fibers as well as to the retention of the curcumin within the FF nanotubes. The present invention therefore provides a usage of sonochemical (ultrasound) irradiation for large-scale fabrication of functionalized commercial fabrics.

Fibers Coated with Self-Assembled Peptide-Based Nanostructures:

According to an aspect of embodiments of the present invention, there is provided a composite structure that includes a core fiber and a plurality of peptide-based self-assembled nanostructures (SAPBNSs) attached thereto.

A composite structure presented herein is therefore composed of two basic elements: a core structure (core fiber) and a (single or multiple) coat, whereby the structure as a whole adopts the shape of the core structure.

In the context of embodiments of the resent invention, the term “core fiber” refers to a single monolithic fiber or a thread spun/weaved from a plurality of fibers. The core fiber, according to embodiments of the present invention, can be spun/woven from natural fibers such as cotton, linen, jute, flax, ramie, sisal and hemp, spider silk, sinew, hair, wool and asbestos (the only naturally occurring mineral fiber). The core fiber may also be spun/woven from man-made synthetic fibers such as fiberglass, rayon, acetate, modal, cupro, lyocell, nylon, polyester, acrylic polymer fibers, polyacrylonitrile fibers and carbon fiber. Preferably, the fiber is made of cotton, or a cotton/synthetic fiber combination.

The core fibers in the composite structures provided herein may also be formed from biodegradable materials, as these are known in the art. According to some embodiments of the present invention, the core fiber is made of a polymeric material, and the polymeric core structure can be either degradable or non-degradable (durable).

Thus, according to some embodiments of the present invention, the composite structure includes a polymeric core structure made of biodegradable or non-degradable polymers and/or biodegradable or non-biodegradable co-polymers.

The core structure is the part of the composite structure that bequeaths most of its mechanical and morphologic properties, having been produced by well-established techniques, which are designed to give the core structure the desired mechanical and morphologic properties.

Fabrics and meshes used as the core structure of the composite structures provided herein can be tailored made so as to provide the composite with the desired properties, selected in accordance with its intended use. The fabrics and meshes can thus be prepared while controlling the characteristics thereof. Alternatively, commercially or otherwise available fabrics and meshes can be utilized as the core in the composite structure described herein. Such fabrics and meshes can be utilized as is or can be subjected to surface treatment prior to use.

The composite structures provided herein can be identified, for example, by electron microscopy, as demonstrated in the Examples section presented below. As can be seen in such electron microscopy images, the surface of the core fiber, which has been free of any significant appendages prior to the sonochemical process, becomes decorated with the nanostructures on at least a part thereof. The density of coverage of the surface depends on the parameters of the sonochemical process, as discussed hereinbelow.

The degree of attachment of the nanostructure to the core fiber, can be assessed by a simple wash treatment, wherein the plurality of SAPBNSs attached to the fiber (or fabric) by a sonochemical process are substantially resistant to reparative washing by an aqueous solution. In other words, once the SAPBNSs, e.g., nanotubes, have been attached to the fiber, there removal cannot be achieved by reparative washing.

### Self-Assembled Peptide Nanostructures:

The fabric provided herein include fibers which are coated, decorated, or otherwise have a plurality of peptide-based nanostructures attached thereto. In some of any of the embodiments described herein, the nanostructures are formed (self-assembled) by peptides. The self-assembled, peptide-based nanostructures used in the context of some embodiments of the present invention, are referred to herein by the acronym “SAPBNS” or “SAPBNSs”. In case the nanostructures are preferably nanotubes, the acronym used may be “SAPBNT” or “SAPBNTs”.

In the context of some embodiments of the present invention, the term “nanostructure” refers to any nano-scale 2-dimensional or 3-dimensional structure, selected from the group including, without limitation, nanotubes, gradient multilayer nanofilm (GML nanofilm), nanocochleates, nanocages, nanofabrics, nanofibers, nanoplatelets, nanoribbons, nanorings, nanorods, and nanosheets, and any combination thereof. Preferably the nanostructures are nanotubes.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to, N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba-bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time.

As used herein throughout, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. The term “amino acid” as used herein includes both D- and L-amino acids.

By “self-assembled peptides” it is meant that the peptides forming the nanostructures (e.g., nanotubes) are such that are capable to self-assemble into structures when subjected to suitable environmental conditions. It is to be understood that self-assembly means that the peptides assemble to form ordered structures without being subjected to chemical reactions (e.g., reactions which lead to covalent bond formation).

Previous studies have shown that short peptides having one or more aromatic amino acid residues, as defined hereinunder, self-assemble into ordered nanostructures, e.g., nanotubes (SAPBNTs), when diluted, heated and cooled in an aqueous solution.

In some of any of the embodiments described herein, the plurality of self-assembled peptides forming the SAPBNSs/SAPBNTs include, or consist of, peptides of 2-6 amino acid residues, wherein at least one of the amino acids in each of such peptides is an aromatic amino acid residue. Such peptides are also referred to herein as aromatic peptides.

Thus, hereinthroughout, the phrase “aromatic peptide” encompasses a plurality of peptides, being the same or different from one another, wherein at least a portion (e.g., 50%, or 60%, or 70%, or 80%, or 90%, or 95%, or 98%, or 99% or all) of the peptides are each independently a peptide of 2-6 amino acid residue, in which at least one of the amino acid residues is an aromatic amino acid residue as described herein.

Each of the aromatic peptides can independently include 1, 2, 3, 4, 5 or 6 aromatic amino acid residues, as described herein.

In some embodiments, each of the peptides in the plurality of peptides is independently an aromatic peptide, as described herein.

In some embodiments, at least 50%, or at least 60%, or at least 70%, or at least 80%, or at least 90%, or at least 95%, or at least 98%, or at least 99% or all of the peptides in the plurality of peptides are the same aromatic peptides, as described in any of the related embodiments herein.

The phrase “aromatic amino acid residue”, as used herein, refers to an amino acid residue that has an aromatic moiety in its side-chain.

As used herein, the phrase “aromatic moiety” describes a monocyclic or polycyclic moiety having a completely conjugated pi-electron system. The aromatic moiety can be an all-carbon moiety or can include one or more heteroatoms such as, for example, nitrogen, sulfur or oxygen. The aromatic moiety can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine, as defined herein.

Exemplary aromatic moieties include, for example, phenyl, biphenyl, naphthalenyl, phenanthrenyl, anthracenyl, [1,10]phenanthrolinyl, indoles, thiophenes, thiazoles and, [2,2′]bipyridinyl, each being optionally substituted. Thus, representative examples of aromatic moieties that can serve as the side chain within the aromatic amino acid residues described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine. In some embodiments, one or more of the peptides in the plurality of peptides is an aromatic dipeptide.

In some embodiments, at least a portion (e.g., at least 50%, or at least 60%, or at least 70%, or at least 80%, or at least 90%, or at least 95%, or at least 98%, or at least 99%) or all, of the peptides in the plurality of peptides are aromatic dipeptides, namely, are peptides of 2 amino acid residues, at least one of the amino acid residues being an aromatic amino acid residue as defined herein.

In some embodiments, each peptide in the plurality of peptides is an aromatic dipeptide.

Herein, an aromatic dipeptide describes a peptide composed of two amino acid residues, wherein at least one of these amino acid residues is an aromatic amino acid residue.

The aromatic dipeptides according to any of these embodiments can be the same or different (e.g., the plurality of peptides comprises two or more types of chemically-distinct aromatic dipeptides). When the aromatic dipeptides are different, they can differ from one another by the type of a non-aromatic amino acid residue and/or by the time of the one or two aromatic amino acid residues.

In some of any of the embodiments of the present invention, at least one peptide in the plurality of peptides used for forming the nanostructures (e.g., nanotubes) is an aromatic dipeptide, comprising two aromatic amino acid residues. In some embodiments, each peptide in the plurality of peptides is an aromatic dipeptide, comprising two aromatic amino acid residues.

Thus, the peptides used for forming the SAPBNSs/SAPBNTs can be dipeptides composed of one or two aromatic amino acid residues.

The aromatic amino acid residues composing the dipeptide can be the same, such that the dipeptide is a homodipeptide, or different. Preferably, the SAPBNSs or SAPBNTs are formed from aromatic homodipeptides.

Hence, according to the presently most preferred embodiment of the present invention, each peptide in the plurality of peptides used for forming the SAPBNSs or SAPBNTs is a homodipeptide composed of two aromatic amino acid residues that are identical with respect to their side-chains residue.

Exemplary aromatic homodipeptide include, but are not limited to, phenylalanine-phenylalanine dipeptide (diphenylalanine peptide), naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide.

In some of any of the embodiments described herein, the plurality of aromatic dipeptides comprises a plurality of diphenylalanine peptides. In some embodiments, the plurality of aromatic dipeptides consists of diphenylalanine peptides (Phe-Phe, or FF, dipeptides).

In other embodiments, the peptides in the plurality of peptides comprise 2, 3, 4, 5 or 6 amino acid residues, or any combination thereof.

In some embodiments, one or more, or each, of the peptides in the plurality of peptides, comprise, in addition to an aromatic amino acid residue(s), an RGD sequence.

As used herein and in the art, an RGD sequence is a sequence of the amino acid residues Arg-Gly-Asp. Analogs or peptidomimetics, as defined herein, of the RGD sequence are also contemplated.

In some of any of these embodiments, a peptide or peptidomimetic which comprises a RGD sequence consists of an RGD sequence and 1, 2 or 3 additional amino acid residues as described herein, at least one of these amino acid residues being an aromatic amino acid residue, as described herein.

In some embodiments, the peptide comprises an RGD sequence and an aromatic amino acid residue as described herein, such that the peptide is a tetrapeptide.

In an exemplary embodiment, the plurality of peptides comprises, or consists of, peptides having the amino acid sequence FRGD.

In any of the embodiments described herein, one or more of the aromatic peptides comprise an end-capped moiety, and can be referred to as an end-capping modified peptide.

The phrase “end-capping modified peptide”, as used herein, refers to a peptide which has been modified at the N-(amine)terminus and/or at the C-(carboxyl)terminus thereof. The end-capping modification refers to the attachment of a chemical moiety to the terminus, so as to form a cap. Such a chemical moiety is referred to herein as an end-capping moiety and is typically also referred to herein and in the art, interchangeably, as a peptide protecting moiety or group.

The phrase “end-capping moiety”, as used herein, refers to a moiety that when attached to the terminus of the peptide, provides an end-capping (or modified terminus). The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2. sup.nd ed. 1991) and Harrison et al., “Compendium of Synthetic Organic Methods”, Vols. 1-8 (John Wiley and Sons, 1971-1996).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denoted herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”).

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus end-capping may be modified to an amide group.

Other end-capping modifications of peptides include replacement of the amine and/or carboxyl with a different moiety, such as hydroxyl, thiol, halide, alkyl, aryl, alkoxy, aryloxy and the like, as these terms are defined herein.

In a preferred embodiment of the present invention, some or all of the peptides that comprise the SAPBNSs/SAPBNTs are end-capping modified only at the N-terminus (namely, peptides having an end-capping moiety substituting the N-terminus of the peptide).

End-capping moieties can be classified by their aromaticity. Thus, end-capping moieties can be aromatic or non-aromatic.

Representative examples of non-aromatic end capping moieties suitable for N-terminus modification include, without limitation, formyl, acetyl trifluoroacetyl, tert-butoxycarbonyl, trimethylsilyl, and 2-trimethylsilyl-ethanesulfonyl. Representative examples of non-aromatic end capping moieties suitable for C-terminus modification include, without limitation, amides, allyloxycarbonyl, trialkylsilyl ethers and allyl ethers.

Representative examples of aromatic end capping moieties suitable for N-terminus modification include, without limitation, fluorenylmethyloxycarbonyl (Fmoc). Representative examples of aromatic end capping moieties suitable for C-terminus modification include, without limitation, benzyl, benzyloxycarbonyl (Cbz), trityl and substituted trityl groups.

In a preferred embodiment of the present invention, the end-capping modified peptides are modified by an aromatic (e.g. Fmoc) end-capping moiety.

In some of any of the embodiments described herein, some or all of the peptides forming the SAPBNSs/SAPBNTs are end-capping modified aromatic Phe-Phe, for example, Fmoc-Phe-Phe (Fmoc-FF). Fmoc-FF has the advantage of being natural, non-toxic, relatively chip, and easy to handle peptide.

In some of any one of the embodiments described herein, some or all of the peptides forming the SAPBNSs/SAPBNTs are end-capping modified FRGD peptides, for example, Fmoc-FRGD peptides.

It is noted herein that the SAPBNSs/SAPBNTs are preferably not processed or further untreated in order to maintain their self-assembled structure, and thus are present in the impregnated fiber/fabric essentially in their original shape, at least to the extent of the forces holding the nanostructures. In other words, according to some embodiments of the present invention, chemical bonding is not required or present in order to keep the nanostructures from falling apart before, during or after the sonochemical irradiation. There is also no need for crosslinking the peptides to one-another in the same nanostructure or between nanostructures. Hence, according to some embodiments of the present invention, the SAPBNSs/SAPBNTs are preferably devoid of crosslinks. According to some embodiments of the present invention, the walls and other main structural elements of the SAPBNSs/SAPBNTs are preferably consisting of the peptide molecules, except for bioactive agents that optionally may be engaged therewith; the bioactive agents are essentially not involved in the formation and stability of the SAPBNSs/SAPBNTs. According to some embodiments of the present invention, the SAPBNSs/SAPBNTs are preferably devoid of oligosaccharides.

As demonstrated hereinbelow, the peptide-based nanotubes are capable of engaging other molecules in such a fashion that can be harnessed for the purpose of delivery and slow release of the bioactive agent from the nanostructures (e.g., nanotubes). In the context of the present invention, the term “engaged” refers to the interaction between the nanostructures and the bioactive agent, and should be seen as used interchangeably with the alternative terms referring to the bioactive agent as being “encapsulated”, “entrapped”, “incorporated” or “entangled” in/with/on the SAPBNSs/SAPBNTs.

### Bioactive Agent:

As discussed hereinabove, the self-assembled peptide-based nanostructures (SAPBNSs or SAPBNTs) are capable of carrying a releasable payload, which can comprise a single type of bioactive agent, or a mixture of different bioactive agents. In the context of the present embodiments, the terms “bioactive agent”, and “pharmaceutically active agent” are used interchangeably. In some embodiments the bioactive agent is a drug.

As used herein, the terms “bioactive agent” and “drug” refer to small molecules or biomolecules that alter, inhibit, activate, or otherwise affect a biological mechanism or event. Bioactive agent that can be encapsulated or incorporated into the SAPBNSs/SAPBNTs, according to embodiments of the present invention, include, but are not limited to, antimicrobial agents (including antibiotics, antiviral, antifungal, anti-parasite, anti-protozoan etc.), anti-cancer substances for all types and stages of cancer and cancer treatments (chemotherapeutic, proliferative, acute, genetic, spontaneous etc.), anti-proliferative agents, photosensitizing agents, chemosensitizing agents, anti-inflammatory agents (including steroidal and non-steroidal anti-inflammatory agents and anti-pyretic agents), anti-oxidants, hormones, anti-hypertensive agents, anti-AIDS substances, anti-diabetic substances, immunosuppressants, enzyme inhibitors, neurotoxins, opioids, hypnotics, anti-histamines, lubricants, tranquilizers, anti-convulsants, muscle relaxants and anti-Parkinson substances, antipruritic agents, anti-spasmodics and muscle contractants including channel blockers, miotics and anti-cholinergics, anti-glaucoma compounds, modulators of cell-extracellular matrix interactions including cell growth inhibitors and anti-adhesion molecules, vitamins, vasodilating agents, inhibitors of DNA, RNA or protein synthesis, analgesics, anti-angiogenic factors, anti-secretory factors, anticoagulants and/or anti-thrombotic agents, anesthetics, ophthalmics, prostaglandins, anti-depressants, anti-psychotic substances, anti-emetics, radioactive agents and imaging agents. A more comprehensive listing of exemplary drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

As used herein, the term “small molecule” refers to molecules, whether naturally-occurring or artificially created (e.g., via chemical synthesis), that have a relatively low molecular weight. Typically, small molecules are monomeric and have a molecular weight of less than about 1500 Da. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, are all considered acceptable for use in accordance with the present invention.

Anti-cancer drugs that can be encapsulated and controllably released from the coated fibers, according to some embodiments of the invention include, include but are not limited to Chlorambucil; 3-(9-Acridinylamino)-5-(hydroxymethyl)aniline; Azatoxin; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon beta 1-alpha; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Non-limiting examples of chemotherapeutic agents that can be efficiently delivered by the coated fibers, according to some embodiments of the present invention, include amino containing chemotherapeutic agents such as camptothecin, daunorubicin, doxorubicin, N-(5,5-diacetoxypentyl)doxorubicin, anthracycline, mitomycin C, mitomycin A, 9-amino aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, irinotecan, topotecan, 9-amino camptothecin, paclitaxel, docetaxel, esperamycin, 1,8-dihydroxy-bicyclo [7.3.1]trideca-4-ene-2,6-diyne-13-one, anguidine, morpholino-doxorubicin, vincristine and vinblastine, and derivatives thereof, sulfhydril containing chemotherapeutic agents and carboxyl containing chemotherapeutic agents. Additional chemotherapeutic agents include, without limitation, an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, a gonadotropin-releasing hormone analog, bleomycin, doxorubicin, paclitaxel, 4-OH cyclophosphamide and cisplatinum.

Anti-inflammatory drugs that can be encapsulated and controllably released from the coated fibers, according to some embodiments of the present invention, include but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

Suitable antimicrobial agents, including antibacterial, antifungal, antiprotozoal and antiviral agents, for use in context of the present invention include, without limitation, beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Representative examples of hormones include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing.

Non-limiting examples of analgesic agents that can be efficiently delivered by the coated fibers, according to some embodiments of the present invention, include acetaminophen, alfentanil hydrochloride, aminobenzoate potassium, aminobenzoate sodium, anidoxime, anileridine, anileridine hydrochloride, anilopam hydrochloride, anirolac, antipyrine, aspirin, benoxaprofen, benzydamine hydrochloride, bicifadine hydrochloride, brifentanil hydrochloride, bromadoline maleate, bromfenac sodium, buprenorphine hydrochloride, butacetin, butixirate, butorphanol, butorphanol tartrate, carbamazepine, carbaspirin calcium, carbiphene hydrochloride, carfentanil citrate, ciprefadol succinate, ciramadol, ciramadol hydrochloride, clonixeril, clonixin, codeine, codeine phosphate, codeine sulfate, conorphone hydrochloride, cyclazocine, dexoxadrol hydrochloride, dexpemedolac, dezocine, diflunisal, dihydrocodeine bitartrate, dimefadane, dipyrone, doxpicomine hydrochloride, drinidene, enadoline hydrochloride, epirizole, ergotamine tartrate, ethoxazene hydrochloride, etofenamate, eugenol, fenoprofen, fenoprofen calcium, fentanyl citrate, floctafenine, flufenisal, flunixin, flunixin meglumine, flupirtine maleate, fluproquazone, fluradoline hydrochloride, flurbiprofen, hydromorphone hydrochloride, ibufenac, indoprofen, ketazocine, ketorfanol, ketorolac tromethamine, letimide hydrochloride, levomethadyl acetate, levomethadyl acetate hydrochloride, levonantradol hydrochloride, levorphanol tartrate, lofemizole hydrochloride, lofentanil oxalate, lorcinadol, lornoxicam, magnesium salicylate, mefenamic acid, menabitan hydrochloride, meperidine hydrochloride, meptazinol hydrochloride, methadone hydrochloride, methadyl acetate, methopholine, methotrimeprazine, metkephamid acetate, mimbane hydrochloride, mirfentanil hydrochloride, molinazone, morphine sulfate, moxazocine, nabitan hydrochloride, nalbuphine hydrochloride, nalmexone hydrochloride, namoxyrate, nantradol hydrochloride, naproxen, naproxen sodium, naproxol, nefopam hydrochloride, nexeridine hydrochloride, noracymethadol hydrochloride, ocfentanil hydrochloride, octazamide, olvanil, oxetorone fumarate, oxycodone, oxycodone hydrochloride, oxycodone terephthalate, oxymorphone hydrochloride, pemedolac, pentamorphone, pentazocine, pentazocine hydrochloride, pentazocine lactate, phenazopyridine hydrochloride, phenyramidol hydrochloride, picenadol hydrochloride, pinadoline, pirfenidone, piroxicam olamine, pravadoline maleate, prodilidine hydrochloride, profadol hydrochloride, propiram fumarate, propoxyphene hydrochloride, propoxyphene napsylate, proxazole, proxazole citrate, proxorphan tartrate, pyrroliphene hydrochloride, remifentanil hydrochloride, salcolex, salethamide maleate, salicylamide, salicylate meglumine, salsalate, sodium salicylate, spiradoline mesylate, sufentanil, sufentanil citrate, talmetacin, talniflumate, talosalate, tazadolene succinate, tebufelone, tetrydamine, tifurac sodium, tilidine hydrochloride, tiopinac, tonazocine mesylate, tramadol hydrochloride, trefentanil hydrochloride, trolamine, veradoline hydrochloride, verilopam hydrochloride, volazocine, xorphanol mesylate, xylazine hydrochloride, zenazocine mesylate, zomepirac sodium and zucapsaicin.

Non-limiting examples of photosensitizers include photofrin, photoporphyrin, benzoporphyrin, tookad, antrin, purlytin, foscan, and halogenated dyes disclosed, e.g. in U.S. Pat. Nos. 9,572,881, 9,040,721, 8,962,797, 8,748,446 and EP2850061.

### Fiber/Fabric Functionalization by Sonochemical Irradiation:

As presented herein, fibers of natural origin, such as cotton, or synthetic fibers, such as polypropylene, can be coated with peptide nanotubes, using sonochemical irradiation.

Thus, according to an aspect of some embodiments of the present invention, there is provided a process of producing the composite structure, or the fabric that includes the composite structure; the process is effected by:

providing a solution of the SAPBNSs/SAPBNTs;

contacting the solution that includes the SAPBNSs/SAPBNTs with a fiber or a fabric; and

subjecting the solution to ultrasonic energy.

The term “sonochemical irradiation” hereinafter refers to exposure to sonic power, generally in the power ultrasonic range of frequencies (also referred to herein as or ultrasonic energy). Likewise, the term “sonochemistry” refers to the study or use of sonochemical irradiation. In the context of embodiments of the present invention, the term sonochemical irradiation is used interchangeably with the term “ultrasonic irradiation”.

The exposure of the core fibers to sonochemical conditions in the presence of the SAPBNSs can be controlled, and thereby the amount of SAPBNSs attached to the fibers can be controlled. As can be seen in the example section that follows below, the amount of SAPBNSs attached by sonochemical (ultrasound) irradiation to the core fiber can be controlled also by:
the concentration of SAPBNSs in the aqueous liquid medium of the sonochemical reaction (1-5 mg/ml);
the energy level used in the sonochemical reaction (40-400 Watt); and
the time of exposure to sonochemical (ultrasonic) irradiation (0.1-10 minutes).

As have been described previously elsewhere, and hereinbelow, SAPBNSs/SAPBNTs are prepared by mixing an amount of the peptide in an aqueous medium;

heating the medium to 70-95° C. for at least 20 minutes while mixing in order to allow the peptides to self-assemble in the SAPBNSs/SAPBNTs; and

cooling said medium, preferably to room temperature.

The preparation of the composite structure provided herein can be effected as a one-pot reaction, wherein the SAPBNSs are formed in the same medium and vessel where they are later being attached to the core fiber.

As discussed herein, the SAPBNSs can be impregnated with bioactive agents for their delivery and slow release from the composite structure when put to use as a drug-delivery medical device. In such cases, the bioactive agent is incorporated into the SAPBNSs by adding the bioactive agent to the solution in which the SAPBNSs are formed, thereby forming SAPBNSs having bioactive agents engaged therewith.

### Medical Device:

The composite structure presented herein represents an article, as in an article-of-manufacture, an item or an object.

According to embodiments of the present invention, the composite structure can be a medical device or a part of a medical device, such as its casing, which is prefabricated independently. Medical devices, according to the present invention, include, without limitation, gauze strip/band, gauze pad/tube, a wound dressing, a bandage, an elastic bandage, a stitching thread, a mesh, a surgical suture thread, a suture mesh, a stent, a skin patch, a bandage, a suture anchor, a screw, a pin, a tack, a rod, an angioplastic plug, a plate, a clip, a ring, a needle, a tube, a dental or orthopedic implant, a guided tissue matrix, an aortic aneurysm graft device, an atrioventricular shunt, a catheter, a heart valve, a hemodialysis catheter, a bone-fracture healing device, a bone replacement device, a joint replacement device, a tissue regeneration device, a tumor targeting and destruction device, a periodontal device, a hernia repair device, a hemodialysis graft, an indwelling arterial catheter, an indwelling venous catheter, a pacemaker casing, a pacemaker lead, a patent foramen ovale septal closure device, a vascular stent, a tracheal stent, an esophageal stent, a urethral stent, a rectal stent, a stent graft, a synthetic vascular graft, a vascular aneurysm occluder, a vascular clip, a vascular prosthetic filter, a vascular sheath, a drug delivery port and a venous valve.

It is noted herein that the composite structures presented herein may be based on stand-alone fibers but not necessarily, namely the core fiber may be a stand-alone device or a part thereof, and can be functionalized with the SAPBNTs before becoming a part of the device, or after it has been incorporated into the device. Hence, when the core structure (device) according to some of the present embodiments is composed of or has fibrous elements, the fibers may be pre-coated with the nanostructures (e.g., nanotubes), or functionalized with the nanostructures after formation of the device, in which case the nanostructures may not coat the fibrous elements at the contact point of intercrossing junctions between fibrous elements in the core structure, or in areas in which the fibers are in contact with other elements ion the device, where the fibrous elements are in direct physical contact with each other in each of these junctions or with other structural elements of the device or its manufacturing supports.

A mesh is loosely woven or knitted fabric that has a large number of closely spaced holes. According to some embodiments of the present invention, the composite structures provided herein are used to form a mesh, or alternatively, a fibrous mesh is being functionalized with the SAPBNTs. The term “mesh”, as used herein, refers to a multidimensional semi-permeable structure that has a large number of closely-spaced holes, which is composed of a plurality of elongated and interconnected elements, such as fibers, strands, struts, spokes, rungs made of a flexible/ductile material, which are arranged in an ordered (matrix, circular, spiral) or random fashion to form a two-dimensional sheet or a three-dimensional object.

A fabric, according to the present embodiments, including a mesh, can be formed by weaving, interlacing, interweaving, knotting, knitting, winding, braiding and/or entangling the elongated elements so they come in contact to form a network of nodes or hubs separated by holes or openings. Alternatively, a fabric can be formed by punching, drilling, cutting or otherwise forming the holes in a sheet of the fabric material.

A three-dimensional object can be formed from fabrics or meshes by either forming a think sheet, staking several fabric sheets or by bending a fabric sheet into a hollow or tubular object. Without limitation, exemplary medical devices that may include meshes, include a gauze, a screen, a strainer, a filter, a stent, a wound-dressing and the likes. For example, a stent, such as the widely used medical device in angioplasty, bronchoscopy, colonoscopy, esophagogastroduodenoscopy and to treat restenosis and other cardiovascular conditions, is an example of a three-dimensional mesh of struts which are interconnected in an orderly fashion and shaped into a cylindrical tube. Hence, according to embodiments of the present invention, the mesh-based object can take the form or be shaped so as to have a form such as a sheet, a tube, a sphere, a box and a cylinder, wherein the coated fibers presented herein serve as a structural element therein.

The coating of an entire pre-fabricated core structure such as a mesh as presented herein, is realized in the nodes, junctions, intercrossing, hubs or otherwise the points of contact where individual sub-structural elements meet (referred to herein and encompassed under the phrase “intercrossing junctions”). For example, in the case where the core structure is a mesh, when a mesh is woven from pre-coated fibers, two intercrossing fibrous core elements may not come in full contact with each other when they form a junction since they are separated with at least two coat layers of nanostructures (e.g., nanotubes) sheathing each thereof. In the embodiment of the coated pre-fabricated meshes presented herein, the core elements touch each other via direct physical contact and the entire junction which is formed therebetween is coated as a whole without having a coat material separating the elements. In practice, this feature expresses itself mainly in the way the mesh experiences the gradual degradation when exposed to physiological conditions. In a mesh which is weaved from pre-coated fibers, the mesh may loosen and even come apart when the coating layers thins and dwindles, or in other cases the polymeric coat may swell and cause the element to distance each other causing a deformation of the core structure to some extent, while the pre-fabricated coated meshes do not experience any change due to the erosion or swelling of the coat and thus the mesh or other similar core structure maintains its structural integrity and stability throughout the process of degradation or swelling of the coat.

Meshes can be formed, woven or otherwise fabricated from fibers made of a natural source such as plants, animal and mineral sources, or be synthetically man-made from naturally occurring and/or synthetic substances.

It is expected that during the life of a patent maturing from this application many relevant fabric fibers coated with self-assembled peptide-based nanostructures (SAPBNTs) will be developed and the scope of the term fabric fibers coated with SAPBNTs is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Preparation of FF Nanotubes and Fabric Functionalization Thereby

Lyophilized powder of FF dipeptide (Scheme 1; purchased from Bachem, Switzerland) was dissolved in 1 ml distilled water to a final concentration of 2 mg/ml and heated for 50 minutes to 90° C. while mixing to enable the dipeptide to dissolved into its monomeric state. Thereafter, the solution was cooled gradually to 25° C. to allow the self-assembly and nanotubes formation.

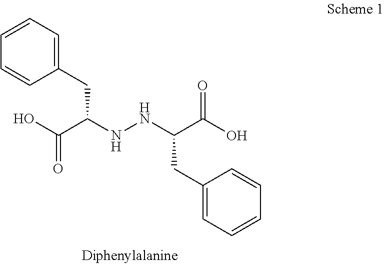 [SMILES: O=C(O)[C@H](Cc1ccccc1)NN[C@@H](Cc1ccccc1)C(=O)O]

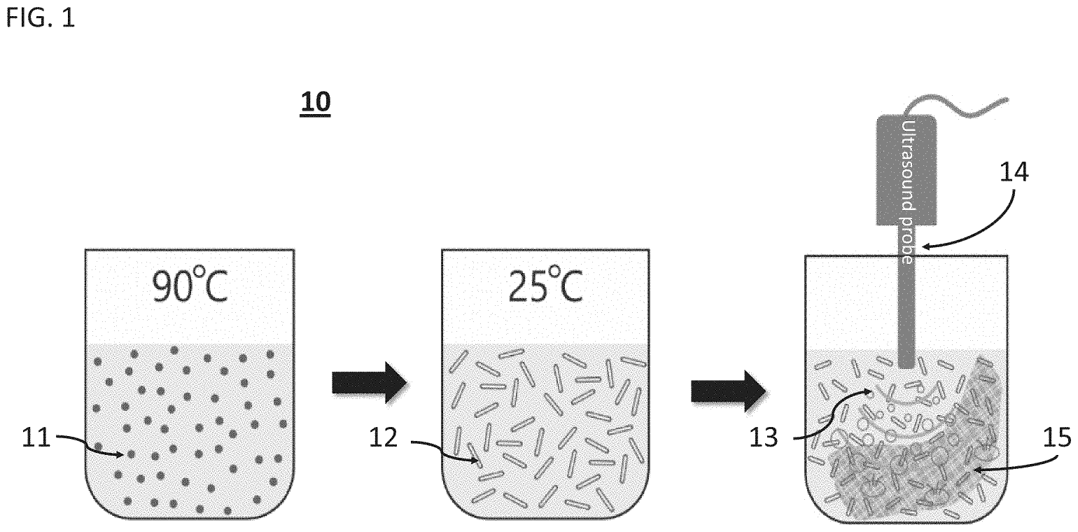

The FF dipeptide nanotubes were dissolved in water by heating to 90° C., and the solution was left to gradually cool down to 25° C. and to allow the self-assembly process into tubular structure, derived by hydrogen bonds, van-der-walls bonds and π-π stacking interactions. A sonochemical process was thereafter applied in order to deposited FF nanotubes onto the fibers of both cotton and non-woven fabrics, as shown in FIG. 1.

FIG. 1 presents a schematic illustration of the process 10 of functionalization of fabrics, according to some embodiments of the present invention, wherein dipeptide FF 11 is incubated at 90° C. and cooled to 25° C. so as to form nanotubes 12, which are then subjected to ultrasound waves 13, emanating from ultrasound probe 14 in the presence of fabric 15.

Cotton gauze pads weighting 129 g/m² (Medicpro, China), and polypropylene-cotton (PP/COT) weighting 11.6 g/m² were kindly provided by AVGOL industries. Experiments were performed on samples with dimensions of 0.5 cm².

Cotton and non-woven fabric sheets of 0.5 cm² immersed into a hot solution of FF dipeptide. The attachment of SAPBNTs to fabrics' fibers was performed with ultrasonic projection with Omni Sonic Ruptor 400 Ultrasonic Homogenizer set to 20% intensity, for various time duration of 30 seconds, 1, 2, 3, 4, 5 minutes after cooling of the solution to room temperature. After completion of the process, the samples were freeze-dried at a temperature of −80° C. for 24 hours and lyophilized overnight.

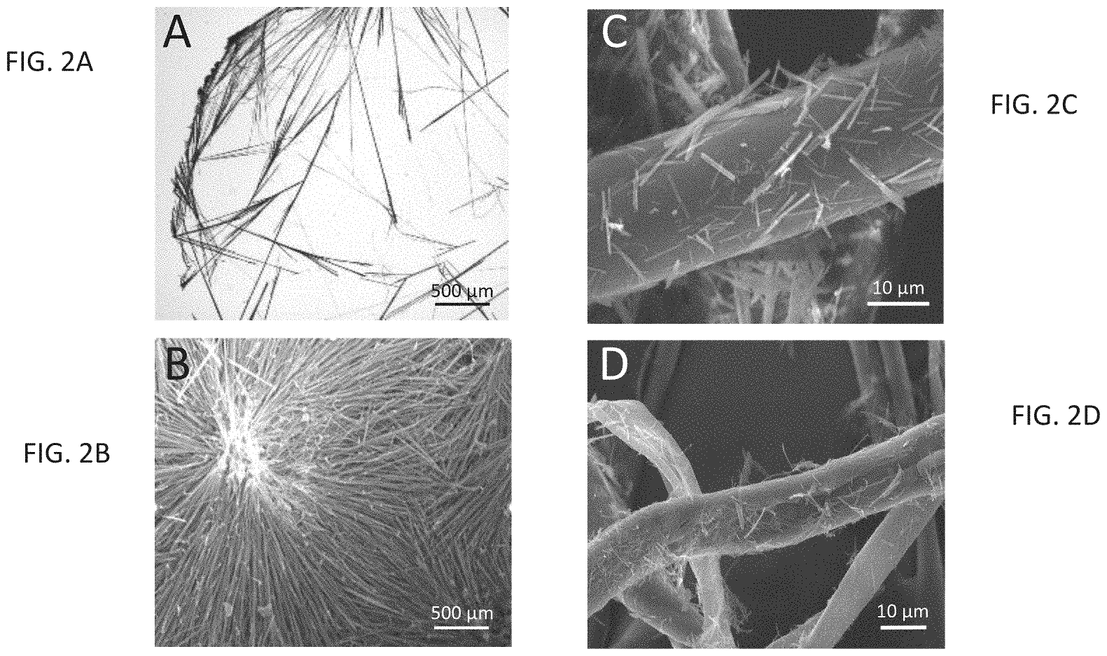

The ordered assemblies were further observed and characterized by an optic and scanning electron microscopy (SEM) (see, FIGS. 2A-D).

To analysed the FF nanostructures and their attachment to the fabrics, samples were observed by JEOL JSM-IT100 plus scanning electron microscope. For this purpose, a dry fabric before and after sonication treatment was mounted to aluminum stab and sputter coated with Cr for conductance.

For the FF dipeptide nanotubes analysis, 10 μl of the solution were deposited on a glass slide and dried in room temperature overnight. The samples sputtered with Cr and imaged using a JSM-6700F High-Resolution Field Emission SEM (Jeol, Japan), operating at an acceleration voltage of 10 Kv.

FIGS. 2A-D present an optic microscope image of FF dipeptide nanotubes (FIG. 2A), a SEM micrograph of FF dipeptide nanotubes (FIG. 2B), a SEM micrograph of FF dipeptide nanotubes deposited on non-woven fabric using sonication irradiation (FIG. 2C), and a SEM micrograph of FF dipeptide nanotubes deposited on cotton fabric using sonication (FIG. 2D).

It is well established that the activity of nanoparticles is greatly depend on their size and shape. As can be seen in FIGS. 2A-D, the microscopic images indicate the size of the structures to be tens of microns in length and thickness of 20-100 nanometers. Some thicker particles can be observed in the images; these large structures are probably bundles of tubes composed of a number of thin tubes. It has been assumed by the present inventors that the bioactivity and release of the bioactive agent from the dipeptide-based nanotubes would be enhanced when their nanotube particle size is in the nanometer scale due to the increased surface to volume ratio of their particle mass. Nevertheless, the shape of the nanoparticles has an impact on their interaction and penetration into bacteria. Moreover, tubular nanostructures are known to allow greater encapsulation efficacy, improved drug delivery throughout the body and better adherence to cells when compared to spherical nanoparticles and liposomes due to a broader surface area in contact with target cells.

For sonochemical functionalization of the fabrics, the dipeptide nanotubes solution were incubated with either the cotton or the non-woven fabric under sonication irradiation to allow the physical binding of the dipeptide nanoparticle to the yarn surface (see, FIG. 1). The sonication were applied using a tip sonicator while cooling the dipeptide solution in an ice-water bath of 0° C. to prevent the overheating of the reactor. It is hypothesized that the adherence of the SAPBNTs to the fabrics is attributed to physical adsorption as a result of the sonication process. It is assumed that as part of the cavitation process, ultrasound waves break the SAPBNTs to shorter particles and forcefully propel them at the fabrics surface, leading to their strong adherence, regardless of their properties. Following the sonication process, the fabrics were lyophilized, and then were observed using SEM (FIGS. 2A-D). SEM images indicate that the FF nanotube homogeneously deposited onto the fabrics.

The dipeptide nanotubes adherence to the fabrics was correlated with sonication time; longer sonication duration of the SAPBNTs with the fabrics facilitated better attachment of the SAPBNTs. Additional increase in sonication time causes aggregation of SAPBNTs to form bundle of fibrils. After 3 minutes of sonication there were no significant elevation in the amount of SAPBNTs attached to the fabric fibers.

The durability of the FF nanotubes coating was assessed following three repeated of manual washings using distilled water for 15 minutes each. Following the washings, the fabrics were lyophilized and imaged in SEM.

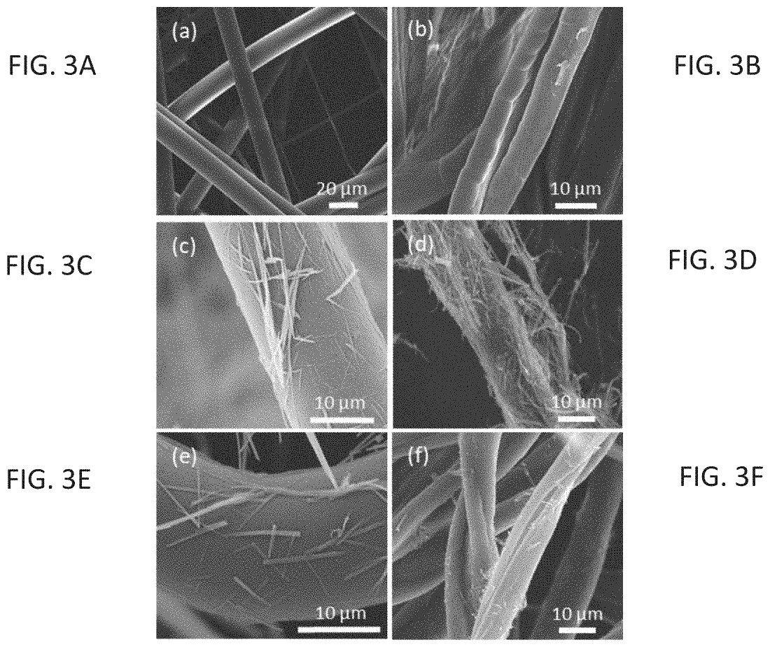

FIGS. 3A-F present SEM images showing the durability of the nanostructure, coated by sonication, to the fabrics, wherein uncoated non-woven fabrics are shown in FIG. 3A, non-woven fabrics coated by FF nanotubes using sonication irradiation are shown in FIG. 3B, non-woven fibers coated by FF nanotubes using sonication irradiation, after washings are shown in FIG. 3C, uncoated cotton fabrics are shown in FIG. 3D, cotton fabrics coated by FF nanotubes using sonication irradiation are shown in FIG. 3E, and cotton fibers coated by FF nanotubes using sonication irradiation, after washings are shown in FIG. 3F.

As can be seen in FIGS. 3A-F, the fabrics preserve their original structure following the sonication process. Moreover, the FF nanotubes can be observed before and after washing, indicating the coating durability. Dipeptide nanotubes durability upon washing is of utmost importance for assessing the performance of the fabrics, in particular when designing antibacterial fabrics.

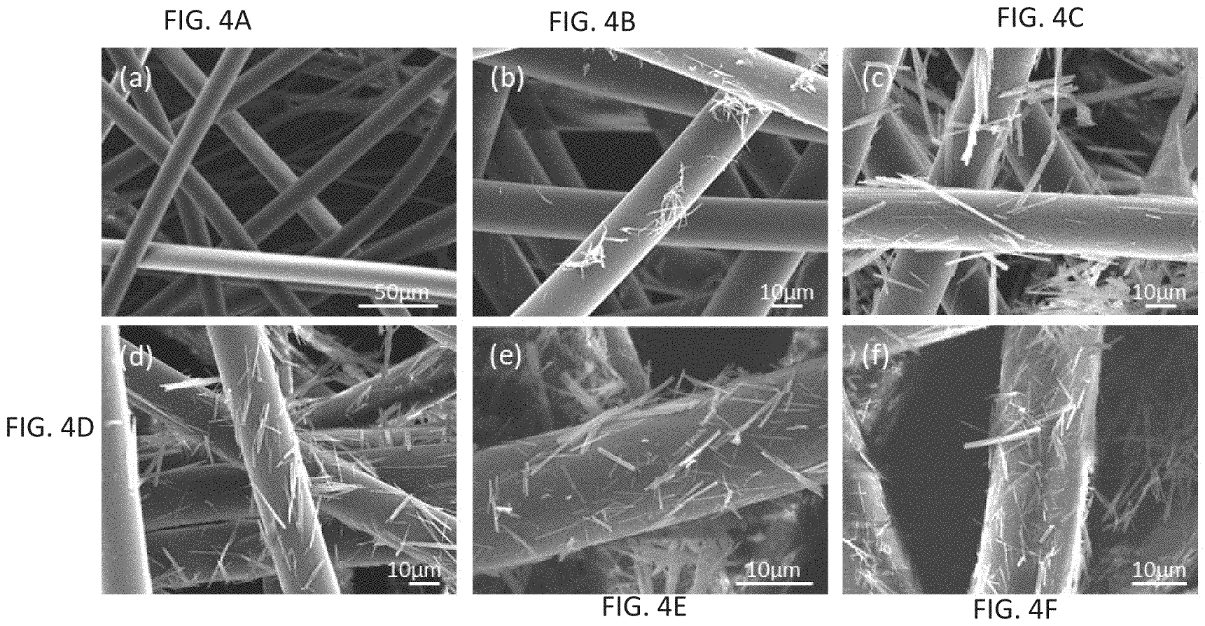

FIGS. 4A-F present SEM images showing the correlation between the degree of SAPBNTs coating of core fibers in synthetic non-woven fabrics as a function of sonochemical (ultrasonic) irradiation time, wherein FIG. 4A shows an unmodified synthetic non-woven fabric, FIG. 4B shows the synthetic non-woven fabric with absorbed nanotubes without sonication, FIGS. 4C-F show the synthetic non-woven fabric coated with nanotubes following sonochemical (ultrasonic) irradiation duration of 30 seconds (FIG. 4C), 1 minute (FIG. 4D), 3 minutes (FIG. 4E), and 5 minutes (FIG. 4F).

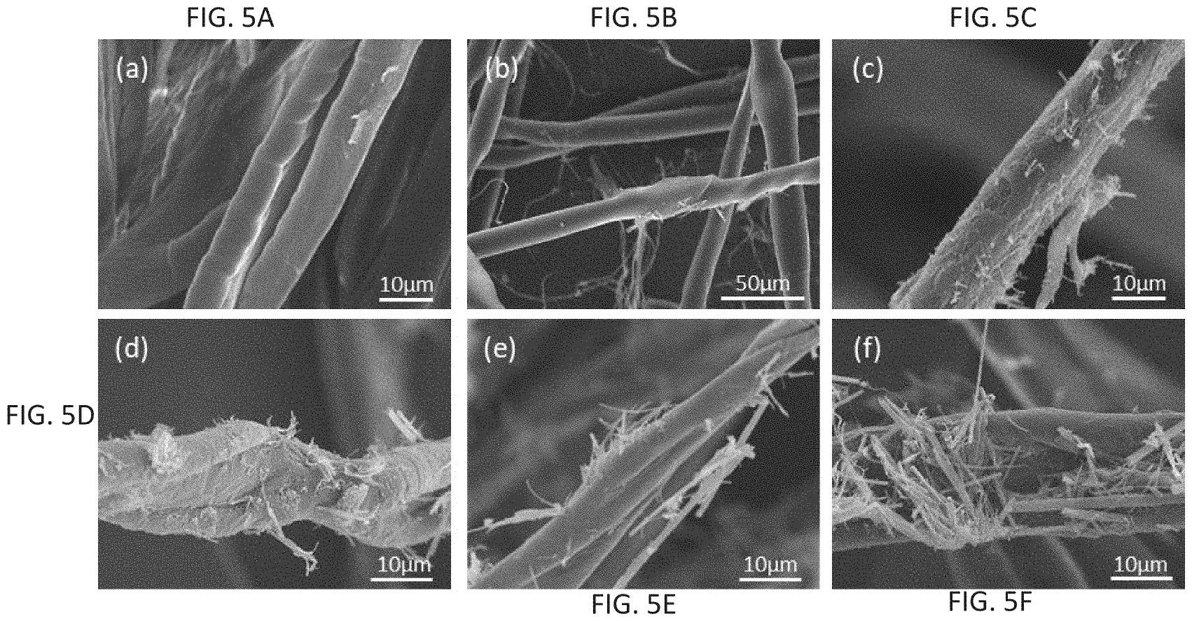

FIGS. 5A-F present SEM images showing the correlation between the degree of SAPBNTs coating of core fibers in 100% cotton fabrics as a function of sonochemical (ultrasonic) irradiation time, wherein FIG. 5A shows an unmodified cotton fabric, FIG. 5B shows the cotton fabric with absorbed nanotubes without sonication, and FIGS. 5C-F show the cotton fabric coated with nanotubes following sonochemical irradiation duration of 30 seconds (FIG. 5C), 1 minute (FIG. 5D), 3 minutes (FIG. 5E), and 5 minutes (FIG. 5F).

### Example 2

### Curcumin Encapsulation in the Dipeptide Nanotubes

The fiber-deposited FF nanotubes were assessed as vehicles for sustained release of bioactive agents, by the encapsulation of curcumin. As a naturally fluorescent molecule due to its polyphenolic structure, curcumin enabled the inventors to track both its encapsulation within the SAPBNTs and its sustained release. The FF nanotubes were synthesized in the presence of curcumin to allow curcumin to undergo encapsulation in the SAPBNTs.

Curcumin ((1E,6E)-1,7-bis(4-hydroxy-3-methoxyphenyl)-1,6-heptadiene-3,5-dione), shown in Scheme 2, was purchased from Sigma-Aldrich, Rehovot.

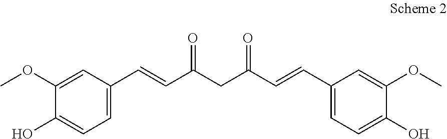

In the context of some embodiments of the present invention, curcumin is an exemplary bioactive agent; it has been reported to exhibit many functions, including anti-inflammatory, anticancer, antiviral, antiarthritic and antioxidant properties. Curcumin can be employed as a bioactive agent, however its poor aqueous solubility, and therefore, minimal bioavailability limit its use. A recent review suggested that the poor clinical performance of curcumin is likely originated from its aggregation and its inherent instability in vivo [Seil, J. T. and Webster, T. J., Int. J. Nanomedicine, 2012, 7, 2767-2781]. Thus, a nanoparticle-based delivery system, such as the exemplary FF nanotubes, serves as a tool for its clinical application due to its ability of sustained release of the exemplary bioactive agent—curcumin.

Curcumin stock solution was prepared by dissolving curcumin powder in ethanol at a concentration of 5 mg/ml. A curcumin solution was added to the desired dipeptide solution at a final concentration of 2 mg/ml. The nanostructures were separated from the solution by centrifugation of 15 minutes at 14,000 rpm.

Fluorescence microscopy analysis of the FF dipeptide nanotube encapsulating curcumin was conducted as follows. 10 μl of the solution containing curcumin encapsulated in FF dipeptide nanotubes were deposited on a glass slide and observed using a Nikon Eclipse Tifluorescent microscope. Images were captured by a ZylascMOS camera using Nikon Intensilight C-HGFI fluorescent lamp and red filter.

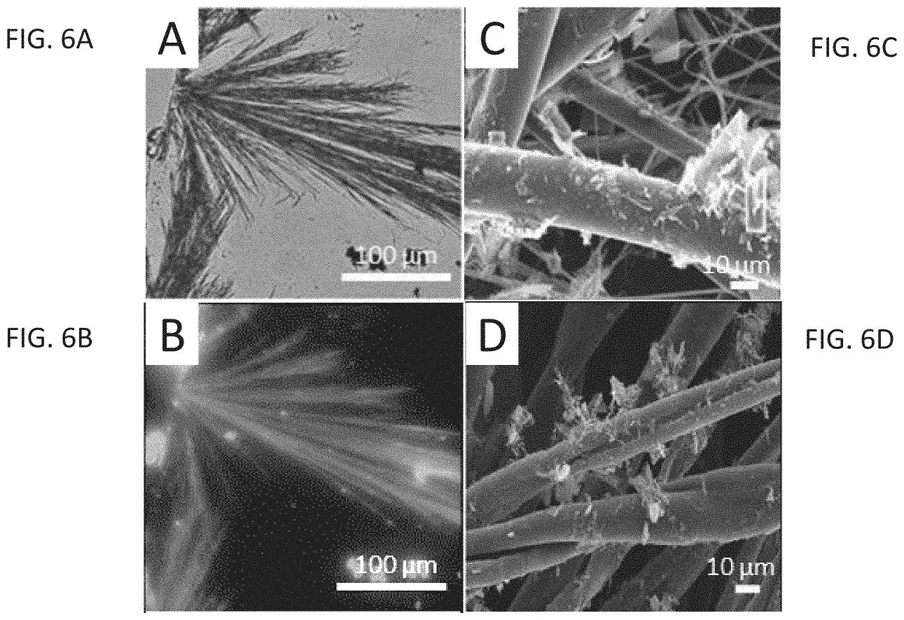

FIGS. 6A-D present SEM micrographs showing optic microscope image of curcumin encapsulation in FF nanotubes (FIG. 6A), fluorescence microscopy of curcumin encapsulated in FF nanotubes (FIG. 6B), non-woven fabric deposited with curcumin loaded FF nanotubes observed by SEM (FIG. 6C), and cotton fabric deposited with curcumin loaded FF nanotubes observed by SEM (FIG. 6D).

As can be seen in FIGS. 6A-D, curcumin encapsulation into the FF nanotubes can be corroborated using an optic and fluorescence microscope, respectively. It can be seen that curcumin loading did not disturb the overall morphology of the SAPBNTs. The fabrics were further placed in the solution of the curcumin loaded FF nanotubes and sonicated to gain physical attachments to the fabrics.

### Example 3

### Curcumin Release from the Fabrics

The kinetic profile of curcumin release from both types of fabrics was monitored using absorbance analysis at 480 nm. Cotton and nonwoven fabrics coated with FF dipeptide nanotubes, encapsulating curcumin, using sonication irradiation were monitored by absorbance at 480 nm measured every 1 min using a TECAN Infinite M200PRO plate reader for a different time frame. The fabric were vertically mounted into a 96-well plate and the measurements performed in ethanol:water (1:1 v/v) solution over time. The absorbance graphs were normalized according to the calibration curves of curcumin solution in ethanol:water (1:1 v/v).

The absorbance characteristics of curcumin were monitored over time and the end point of the measurement was defined when the kinetics of release reached a plateau. Release of free curcumin from the cotton and non-woven fabrics and curcumin encapsulated in FF nanotubes without the attachment to the fabric by sonication was used as a control and compared to the release of curcumin encapsulated in FF nanotubes and attached to the fabrics using sonication for the duration of 30 and 60 seconds.

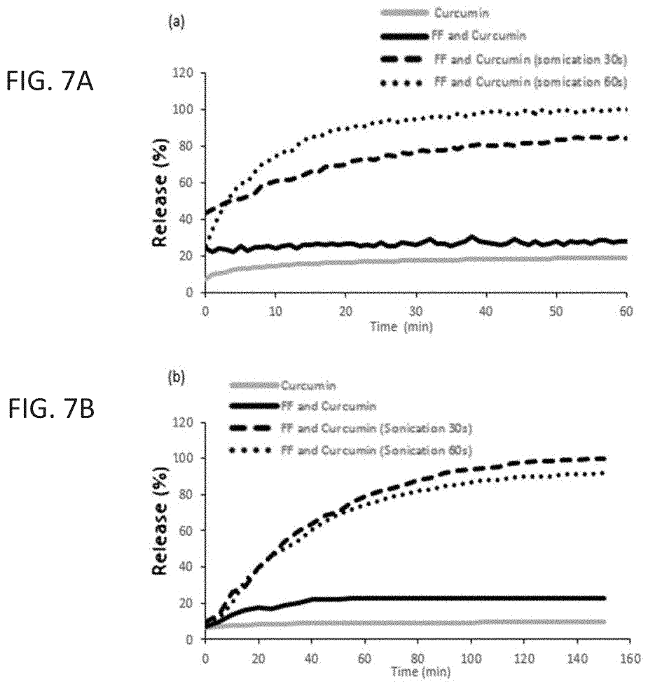

FIGS. 7A-B present curcumin release kinetics profiles, as observed while released from dipeptide nanotubes attached to the fabrics, according to some embodiments of the present invention, wherein absorbance kinetics of curcumin released from FF nanotubes coated non-woven fabric (430 nm) is shown in FIG. 7A, and slow release graph of curcumin from cotton coated by FF nanotubes is shown in FIG. 7B, whereas the curves were normalized according to the calibration curves of curcumin solution.

As can be seen in FIGS. 7A-B, in both control experiments, the amount of absorbed curcumin was very low (about 20%) and it's release occurred immediately upon exposure to the solvent. Curcumin encapsulated in FF nanotubes that were attached to the fabrics using sonication process showed different pattern of release. First, the dipeptide nanotubes, which were attached through sonication, demonstrated high amount of absorbed curcumin and slow release kinetics compared to the non-sonicated reference samples. In the case of non-woven fabric (FIG. 7A), the samples sonicated for 60 seconds absorbed curcumin at 9% of its original weight. The release of 80% of curcumin from this sample occurs within 12 minutes, and reached a plateau after 40 minutes. It can be seen that the release profile of sample sonicated for 30 seconds differs from that of 60 seconds. The curcumin release rate from this sample was almost constant and reached its plateau after 55 minutes. In the case of cotton fabric (FIG. 7B), both samples of 30 seconds and 60 seconds sonication show similar profile. During one hour the release kinetic was constant and reaches 75% of total amount of released curcumin and reached a plateau after 140 minutes.

In summary, the examples presented hereinabove demonstrate that functionalized fabrics, according to embodiments of the present invention, are highly effective for various applications. The dipeptide nanotubes were fully embedded onto the fabrics' fibers and allowed sustained release of the exemplary bioactive agent curcumin, while both SAPBNT adherence and curcumin release were controllably modulated by sonication time.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composite structure comprising a core fiber and a plurality of peptide-based self-assembled nanostructures (SAPBNSs) attached thereto.

2. The composite structure of claim 1, wherein said SAPBNSs comprise at least one peptide having 2-6 amino-acid residues.

3. The composite structure of claim 1, wherein said peptide comprises at least one aromatic amino acid residue.

4. The composite structure of claim 3, wherein said peptide is a dipeptide.

5. The composite structure of claim 4, wherein said dipeptide is a homodipeptide.

6. The composite structure of claim 4, wherein said homodipeptide is selected form the group consisting of phenylalanine-phenylalanine dipeptide (diphenylalanine peptide; FF), naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide.

7. The composite structure of claim 1, wherein said nanostructures are essentially nanotubes.

8. The composite structure of claim 1, wherein said core fiber is a cotton fiber, a wool fiber, a silk fiber, a synthetic fiber and a mixed natural and synthetic fiber.

9. The composite structure of claim 1, wherein said plurality of SAPBNTs is attached to said core fiber as assessed by being substantially resistant to reparative washing by an aqueous solution.

10. The composite structure of claim 1, wherein at least one of said plurality of SAPBNTs comprise a bioactive agent engaged therewith.

11. The composite structure of claim 10, wherein said bioactive agent is curcumin.

12. A fabric comprising at least one fiber of claim 1.

13. The fabric of claim 12, being a woven fabric.

14. The fabric of claim 12, being a non-woven fabric.

15. The fabric of claim 12, consisting of said fiber.

16. A medical device comprising at least one fiber of claim 1, or comprising a fabric that comprises the same.

17. The medical device of claim 16, selected from the group consisting of a gauze, a wound dressing, a stitching thread, a bandage and mesh.

18. A process of producing the fiber of claim 1, comprising:
providing said plurality of peptide-based self-assembled nanostructures in a solution;
contacting said solution that comprises said plurality of peptide-based self-assembled nanostructures with said fiber or said fabric; and
subjecting said solution to sonochemical (ultrasound) irradiation.

19. The process of claim 18, wherein said providing comprises:
mixing said peptide in an aqueous medium;
heating said medium to 70-95° C. for at least 20 minutes while mixing; and
cooling said medium.

20. The process of claim 19, wherein said mixing further comprises adding a bioactive agent to said aqueous medium.

21. The process of claim 18, wherein said sonochemical irradiation is in the range of 40 watt to 400 watt.

22. The process of claim 18, wherein a concentration of said SAPBNSs in said solution ranges 1-5 mg/ml.

23. The process of claim 18, wherein subjecting said solution to sonochemical (ultrasound) irradiation is effected over a period that ranges from 1 second to 10 minutes.

24. A method of treating a medical condition using the medical device of claim 16.
